(19) United States
(12) Patent Application Publication
(10) Pub. No.: US 2005/0096381 A1
(43) Pub. Date: May 5, 2005

(54) Derivatives of isoflavones

(75) Inventors: Fortune Kohen, Tel-Aviv (IL); Batya Gayer, Mazkeret Batya (IL); Naftali Stern, Nir Zvi (IL); Dalia Somjen, Rehovot (IL)

Correspondence Address: WINSTON & STRAWN;PATENT DEPARTMENT, 1400 L STREET, N.W., WASHINGTON, DC, 20005-3502, US

(73) Assignee: YEDA & MEDICAL RESEARCH

(21) Appl. No.: 10/943,943

(22) Filed: Sep. 20, 2004

Related U.S. Application Data
(63) Continuation of application No. PCT/IL03/00224, filed on Mar. 16, 2003.

(30) Foreign Application Priority Data
Mar. 21, 2002 (IL) 148825

(51) Int. Cl. A61K 31/353; C07D 311/74

(52) U.S. Cl. 514/456; 549/403

(57) ABSTRACT

The present invention discloses novel derivatives of isoflavones, in particular carboxy derivatives of isoflavones, active as selective estrogen receptor modulators, and uses of the carboxy derivatives for the treatment of estrogen-related conditions. The present further discloses conjugates of these carboxy derivatives of isoflavones and their use for affinity targeting of drugs, imaging and detection agents to cells having estrogen receptors, particularly estrogen receptors subtype β.

### CROSS-REFERENCE TO RELATED APPLICATIONS

This application is a continuation of International application PCT/IL2003/000224 filed Mar. 16, 2003, the content of which is expressly incorporated herein by reference.

### FIELD OF THE INVENTION

The present invention relates to novel derivatives of isoflavones, in particular to carboxy derivatives of isoflavones capable of binding to estrogen receptors, more particularly to carboxy derivatives of the isoflavones biochanin A, daidzein, formononetin and genistein and their use as selective estrogen receptor modulators, as well as to conjugates of said carboxy derivatives of isoflavones, and their use for affinity targeting to cells having estrogen receptors.

### BACKGROUND OF THE INVENTION

The hormone estrogen has a broad spectrum of effects on tissues in both females and males. Many of these biological effects are positives, including maintenance of bone density, central nervous system function, and the protection of organ systems from the effect of aging. However, in addition to positive effects, as estrogen regulates the function and differentiation of various tissues such as the reproductive system, breast, adrenal or colon (Enmark E & Gustafsson J A 1999 J. Intern. Med. 246: 133-138), it is also known to be associated with cancer in these tissues.

Estrogens mediate their effects via nuclear estrogen receptors ERa or ERGS, which are differentially distributed among tissues, in both normal and malignant cells types (Pettesson K& Gustafsson J A 2001 Annu. Rev. Physiol. 63: 165-192). For instance, the human mammary cancer cell line MCF-7 expresses mainly ERa while human colon, lung and adrenal carcinoma cell lines express mainly ERGS.

Ligands can bind to the two different ERs, which, in the presence of tissue-specific co-activator and/or co-repressors, bind to an estrogen response element in the regulatory region of genes or to other transcription factors. Both subtypes of ERs mediate gene transcription via a classical estrogen response element (ERE) or via an activator protein (AP)-1 enhancer element. Given the complexity of ER signaling, along with the tissue-specific expression of ERa and ERP and their co-factors, it has been recognized that ER ligands can act as estrogen agonists as well as antagonists, and new class of compounds, referred to as Selective Estrogen Receptor Modulators (SERMs) has been discovered.

For example, when an estrogen-receptor complex binds to DNA at a classical ERE site, an estradiol-ER (α or β) complex initiates transcription, while an anti-hormone (e.g. tamoxifen)-ER complex blocks it. If estrogen binding occurs at the AP-1 site, a different mechanism is involved, and in this case the estradiol-ERα complex inhibits transcription while the anti-hormone-ERβ complex activates it. ERβ can, therefore, have opposite effects depending on the DNA binding site (Nilsson S & Gustafsson J A 2000 Breast Cancer Res. 2:360-366).

The two ERs differ also in terms of their ligand binding profiles. Although estradiol display a high binding affinity for both ERs, differences in binding affinity were noted with respect to estrogen antagonists (e.g. raloxifene), xenoestrogens and isoflavones.

Isoflavones are phytochemicals having molecular weights and structures similar to steroids. Foods containing soy proteins are a rich source of isoflavone phytoestrogens, such as genistein and daidzein. These substances gained increased attention as lower rate of chronic diseases, including coronary heart disease, and reduced incidence of breast, prostate and colon cancer have been associated with high dietary intake of soy-containing foods. Soy phytoestrogens bind weakly to estrogen receptors, and some, for example genistein, bind more strongly to ERβ than to ERα. The isoflavones display both weak estrogenic and anti-estrogenic properties, and they can therefore be considered as SERMs.

The inventors of the present invention have previously shown the synthesis of isoflavone derivatives by introducing a carboxy group at position 6 or 7 of the isoflavone molecule, for the generation of monoclonal antibodies to isoflavones (Kohen F. et al. 1999 Nutr. Cancer 35:96-10; Kohen F. et al. 1998 J. Steroid Biochem. Mol. Biol. 64:217-222) valuable as research tools for measuring isoflavone levels in human urine after soy digestion.

In addition to the estrogenic and anti-estrogenic effects, isoflavones show a wide spectrum of biological activities. Genistein, shown to inhibit the protein-tyrosine kinase pathway, was used in a treatment of choroidal neovascularization (U.S. Pat. No. 6,028,099). Genistein was also shown to have activity as topoisomerase II, and to induce apoptosis and cell differentiation. Moreover, genistein has been shown to inhibit the proliferation of both cancer and normal cells, and was used for prophylactic treatment of cataract (WO 00/37066).

The 4′methoxy derivative of genistein, biochanin A, is equally potent to genistein as a growth inhibitor in breast cancer lines due to its conversion to genistein (Peterson et al. 1998 Am. J. Clin. Nutr. 68:1505-1511S). In addition, when administered in equal doses, biochanin A, and not genistein, inhibited the growth of several tumors derived from the gastrointestinal tract and grown in nude mice.

Chemotherapy constitutes one of the major therapeutic approaches for the treatment of cancer, along with surgery and radiotherapy. However, the usefulness of commonly used anti-cancer drugs such as daunomycin and adriamycin is severely limited by their toxicity towards normal tissues, particularly the myocardium and the rapidly proliferating cells of the gastrointestinal tract and bone marrow. In addition, these drugs are affected by the mechanisms of multi-drug resistance. Affinity targeting of these cytotoxic drugs to tumor cells offers an approach that might overcome some of these drawbacks. In recent years monoclonal antibodies, proteins or peptide hormones for which specific receptors are located on membranes of tumor cells have been used as carriers or targetors of cytotoxic drugs. This approach has been exemplified by the use of analogs of luteinizing hormone releasing hormone (LHRH) (Nagy A. et al. 1996 Proc. Natal. Acad. Sci. USA 93:7269-7273), growth factors (WO 88/00837) or melanocyte-stimulating hormone (MSH) (Varga J M et al. 1977 Nature 276:56-58) conjugated to cytotoxic drugs for targeted chemotherapy of cancers that possess membranal receptors. On the other hand, site directed chemotherapy utilizing nuclear receptors (e.g. estrogen receptor) is not well documented. In fact, few studies have been described on the use of estrogen-cytotoxic drug conjugates (e.g. Estracyt, Leo 299; Heiman et al. 1980 J. Med. Chem. 23:994-1002) for affinity therapy, and success with such steroid-drug conjugates has been rather limited.

Thus, there is a recognized need for, and it would be highly advantageous to have improved, ERβ-specific SERMs, which can be used for affinity drug targeting.

### SUMMARY OF THE INVENTION

It is an object of the present invention to provide carboxy derivatives of isoflavones capable of binding to estrogen receptors.

It is another object of the present invention to provide carboxy derivatives of isoflavones active as selective estrogen receptor modulators.

It is yet another object of the present invention to provide isoflavone conjugates.

It is a further object of the present invention to provide methods of using said isoflavone conjugates for affinity targeting to cells having estrogen receptors (ER).

According to one aspect, the present invention relates to carboxy derivatives of isoflavone, active as SERMs.

According to one embodiment, the present invention provides an isoflavone derivative having the general formula (1): 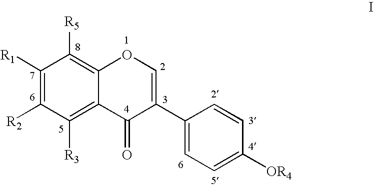
wherein
R₁ is selected from the group consisting of OH, OCH₃ OGlc and OR′COOX;
R₂ is selected from the group consisting of H and R′COOX;
R₃ is selected from the group consisting of H, OH, R′COOX and OR′COOX;
R₄ is selected from the group consisting of H, CH₃ and R′COOX;
R₅ is selected from the group consisting of H and R′COOX;
R′ is selected from the group consisting of (C₁-C₆)alkyl, (C₁-C₂₀)alkoxy, (C₁-C₂₀) alkenyl;
X is selected from the group consisting of H and (CH₂)n-Y wherein Y is CH₃ or NH₂ and n=0-10;
   with the proviso that at least one of R₁, R₂, R₃, R₄ or R₅ comprises a carboxy group.

The present invention discloses the estrogen-like activity of the carboxy derivatives of the isoflavones, which, unlike the underivatized parent isoflavones, display estrogen antagonist properties. Moreover, the carboxy derivatives of the isoflavones have unexpected advantages compared to the parent molecules in terms of their efficacy compared to known SERMs.

Currently preferred carboxy-derivatives according to the present invention are selected from the group consisting of 6-carboxymethyl biochanin A, 8-carboxymethyl biochanin A, 7-(O)-carboxymethyl daidzein, 7-(O)-carboxymethyl formononetin and 6-carboxymethyl genistein. Currently most preferred are 6-carboxymethyl biochanin A and 7-(O)-carboxymethyl formononetin.

According to another aspect, the present invention relates to isoflavone conjugates, specifically to isoflavone conjugated to a drug or to a diagnostic agent.

According to one embodiment, the present invention provides isoflavone conjugates having the general formula (II): 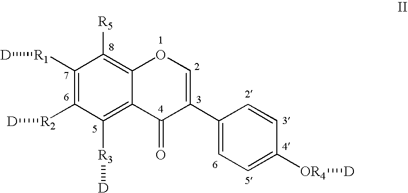
wherein
R₁ is selected from the group consisting of OH, OCH₃ OGlc, OR′COOX and OR′CO;
R₂ is selected from the group consisting of H, R′COOX and R′CO;
R₃ is selected from the group consisting of H, OH, R′COOX, R′CO, OR′COOX and OR′CO;
R₄ is selected from the group consisting of H, CH₃, R′COOX and R′CO;
R₅ is selected from the group consisting of H, R′COOX and R′CO;
R′ is selected from the group consisting of (C₁-C₆)alkyl, (C₁-C₂₀)alkoxy, (C₁-C₂₀) alkenyl;
X is selected from the group consisting of H and (CH₂)n-Y wherein Y is CH₃ or NH₂ and n=0-10;
D may be absent or is a bioactive moiety;
   with the proviso that at least one of R₁, R₂, R₃, R₄ or R₅ is conjugated to D.

According to one embodiment, D is selected from the group consisting of a cytotoxic compound, a cytostatic compound, an antisense compound, an anti-viral agent, a specific antibody, an imaging agent and a biodegradable carrier. It is to be understood that the present invention explicitly excludes all known isoflavone conjugates including 7-(O)-carboxymethyl daidzein-Keyhole Limpet Hemocyanin (KLH), 7-(O)-carboxymethyl daidzein-ovalbumin, 6-carboxymethyl genistein-Horseradish peroxidase (HRP) and 6-carboxymethyl genistein-KLH.

According to another embodiment, the cytotoxic compound D is selected from, but not restricted to agents inhibitory of DNA synthesis and function: adriamycin, bleomycin, chlorambucil, cisplatin, daunomycin, ifosfamide and melphalan; agents inhibitory of microtubule (mitotic spindle) formation and function: vinblastine, vincristine, vinorelbine, paclitaxel (taxol) and docetaxel; anti metabolites: cytarabine, fluorouracil, fluroximidine, mercaptopurine, methotorexate, gemcitabin and thioquanine; alkylating agents: mechlorethamine, chlorambucil, cyclophosphamide, melphalan and methotrexate; antibiotics: bleomycin and mitomycin; nitrosoureas: carmustine (BCNU) and lomustine; inorganic ions: carboplatin, oxaloplatin; interferon and asparaginase; hormones: tamoxifen, leuprolide, flutamide and megestrol acetate.

According to one preferred embodiment, the cytotoxic substance D is an anti-tumor agent.

According to one currently preferred embodiment the anti-tumor agent is daunomycin, and the carboxy-isoflavone is selected from the group consisting of 6-carboxymethyl biochanin A, 8-carboxymethyl biochanin A, 7-(O)-carboxymethyl daidzein, 7-(O)-carboxymethyl formononetin and 6-carboxymethyl genistein.

According to another preferred embodiments, D is an imaging agent selected from, but not restricted to paramagnetic particles: gadolinium, yttrium, lutetium and gallinum; radioactive moieties: radioactive indium, rhenium and technetium; and dyes: fluorescein isothiocyanate (FITC), green fluorescent protein (GFP), Cyan fluorescent protein (CFP), rhodamine I, II, III and IV, rhodamine B, and rosamine.

In another aspect of the embodiment, a plurality of bioactive moieties (D) are conjugated to at least two of R₁, R₂, R₃, R₄ or R₅, wherein D may be the same or different at each occurrence.

According to one preferred embodiment, a plurality of bioactive moieties D are conjugated to at least two of R₁, R₂, R₃, R₄ or R₅, wherein at least one D is a therapeutic agent and at least one D is a biodegradable carrier.

According to one currently preferred embodiment at least one D is a polyvalent natural or synthetic peptide or polypeptide, having free carboxy or amino groups.

According to yet another aspect the present invention relates to pharmaceutical compositions comprising as an active ingredient a carboxy derivative of isoflavone and a pharmaceutically acceptable diluent or carrier.

According to a further aspect the present invention relates to pharmaceutical compositions comprising as an active ingredient an isoflavone conjugate and a pharmaceutically acceptable diluent or carrier.

According to yet further aspect the present invention relates to a method comprising the step of administering to a subject in need thereof a therapeutically effective amount of an isoflavone derivative as an estrogen receptor modulator.

According to one further aspect the present invention relates to a method for site directed chemotherapy using a cytotoxic isoflavone conjugate for affinity drug targeting to an estrogen receptor, preferably estrogen receptor subtype β.

According to one embodiment the present invention relates to a method for site directed chemotherapy using an isoflavone conjugate comprising a cytotoxic agent with or without a biodegradable carrier for affinity drug targeting to an estrogen receptor, preferably estrogen receptor subtype β.

According to yet another aspect, the present invention relates to a method for diagnosis of tumors and other disorders using a labeled isoflavone conjugate for affinity label targeting to an estrogen receptor, preferably estrogen receptor subtype β.

According to one embodiment, the present invention relates to a method comprising the step of administering to a subject in need thereof a therapeutically effective amount of a pharmaceutical composition comprising as an active ingredient a carboxy derivative of isoflavone or a cytotoxic isoflavone conjugate.

According to another embodiment the present invention relates to a method comprising the step of administering to a subject in need thereof a diagnostically effective amount of pharmaceutical composition comprising as an active ingredient a labeled isoflavone conjugate.

According to one preferred embodiment the present invention relates to a method for diagnosing or treating a disorder selected from the group consisting of cancer (e.g. breast, prostate and colon), cardiovascular diseases, osteoporosis, Alzheimer's disease and arteriosclerosis.

The present invention is explained in greater detail in the description, Figures and claims below.

### BRIEF DESCRIPTION OF THE DRAWINGS

FIG. 1 shows synthesis and structure of carboxy derivatives of isoflavones. (A) Synthesis of 6-carboxymethyl biochanin A and 8-carboxymethyl biochanin A. (B) Structures of 6-carboxymethyl genistein and 7-O-carboxymethyl daidzein.

FIG. 2 shows the structures of 6-carboxymethyl biochanin A daunomycin conjugate, 6-carboxymethyl genistein daunomycin conjugate and 7-O-carboxymethyl daidzein daunomycin conjugate.

FIG. 3 demonstrates dose dependent inhibition of DNA synthesis in human vascular smooth muscle cells (VSMC) by cytotoxic isoflavone conjugates as assessed by [³H]thymidine incorporation. Results are means±SD of 3 to 9 replicates. The 50% inhibition for daunomycin as a control, and for 6-carboxymethyl genistein daunomycin conjugate and 7-(O)-carboxymethyl daidzein daunomycin conjugate in these cells is shown as a dashed line on the x-axis.

FIG. 4 demonstrates dose dependent inhibition of DNA synthesis in adrenocortical carcinoma cells (NCI-H295R) by cytotoxic isoflavone conjugates as assessed by [3H]thymidine incorporation. Results are means±SD of 3 to 9 replicates. The 50% inhibition of DNA synthesis for daunomycin as control and for 6-carboxymethyl genistein daunomycin conjugate is shown as a dashed line on the x-axis.

### DETAILED DESCRIPTOIN OF THE INVENTION

The present invention relates to isoflavone derivatives, more specifically to carboxy derivatives of isoflavone, capable of binding to estrogen receptors. The present invention also relates to carboxy derivatives of isoflavones active as selective estrogen receptor modulators.

The present invention further relates to isoflavone conjugates, capable of targeting cytotoxic or diagnostic agents to cell bearing estrogen receptors, located within the cell cytoplasm.

According to one aspect, the present invention relates to carboxy derivatives of isoflavone, active as SERMs.

According to one embodiment, the present invention provides an isoflavone derivative having the general formula (I): 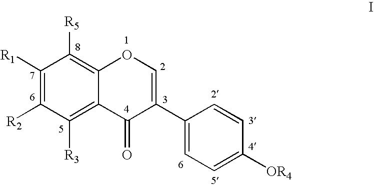
wherein
R₁ is selected from the group consisting of OH, OCH₃ OGlc and OR′COOX;
R₂ is selected from the group consisting of H and R′COOX;
R₃ is selected from the group consisting of H, OH, R′COOX and OR′COOX;
R₄ is selected from the group consisting of H, CH₃ and R′COOX;
R₅ is selected from the group consisting of H and R′COOX;
R′ is selected from the group consisting of (C₁-C₆)alkyl, (C₁-C₂₀)alkoxy, (C₁-C₂₀) alkenyl;
X is selected from the group consisting of H and (CH₂)n-Y wherein Y is CH₃ or NH₂ and n=0-10;
   with the proviso that at least one of R₁, R₂, R₃, R₄ or R₅ comprises a carboxy group.

As used herein, the term “alkyl” denotes branched or unbranched hydrocarbon chains, such as methyl, ethyl, n-propyl, iso-propyl, n-butyl, sec-butyl, iso-butyl, tertbutyl, 2-methylpentyl and octa-decyl. The term “alkoxy” denotes —OR, wherein R is alkyl. The term “alkenyl” denotes branched or unbranched hydrocarbon chains containing one or more carbon-carbon double bonds. The term “Glc” denotes glucosyl or glucoside.

The present invention discloses the use of the isoflavone ring as a template for designing isoflavone carboxy derivatives useful as SERMs based on the following considerations:
(i) The phenolic hydroxyl group of the isoflavone molecule can mimic the 3-OH group of estradiol, and interact through H-bonding with Arg 353 and Glu 394 of the estrogen receptor ERα or Arg 346 and Glu 305 of the estrogen receptor ERβ.
(ii) The hydroxyl group or any acidic substituent of the isoflavone ring can mimic the 17β-OH of estradiol and can form a hydrogen bond with His 524 of the ERα or His 475 of the ERβ.
(iii) Isoflavones (e.g. genistein, daidzein and biochanin A) have been reported to have weak estrogenic and anti-estrogenic properties and biochanin A can serve as a prodrug scaffold (Peterson T G et al. 1998 Am. J. Clin. Nutr. 68:1505S-1511S).
(iv) The new generation of ER antagonists such as GW7604, a tamoxifen derivative, have acidic moieties instead of a basic group in their protruding side chain.

Based on these considerations the present invention now discloses introducing a carboxy group on the isoflavone ring, using an alkyl, alkoxy or alkenyl bridging group, further discloses the resulted carboxy-derivatives of isoflavones as novel SERMs, possessing mixed agonist/antagonist estrogenic properties. More particularly, the present invention discloses the estrogenic and anti-estrogenic properties of 6-carboxymethyl biochanin A, 8-carboxymethyl biochanin A, 7-(O)-carboxymethyl daidzein, 7-(O)-carboxymethyl formononetin and 6-carboxymethyl genistein, tested in vitro for their estrogenic activity and in vivo for their mixed agonist/antagonist activity.

The ability of the isoflavone derivatives to bind estrogen receptor and/or to modulate estrogen receptor response may be examined by any assay known in the art. A convenient assay described herein as a non-limiting example utilizes the specific activity of creatine kinase (CK), an estrogen responsive enzyme, as a parameter for the estrogen-like activity of the isoflavone derivatives of the present invention.

As exemplified herein below, 6-carboxymethyl genistein and 6-carboxymethyl biochanin A caused an increase in CK activity in rat tissues, e.g aorta, diaphysis, epiphysis, left ventricle of the heart and pituitary, with the exception of the uterus. Moreover, the carboxymethyl derivatives of the isoflavones have unexpected advantages compared to the parent molecules in terms of efficacy, being superior to known SERMs. 6-carboxymethyl genistein and 6-carboxymethyl biochanin A blocks the stimulatory effect of estrogen (E2) on creatine kinase (CK) specific activity at 2 to 10 fold lower concentrations compared to the known SERM raloxifene, in tissues derived from both immature and ovariectomized female rats.

According to another aspect, the present invention relates to pharmaceutical compositions comprising the isoflavone derivatives of the present invention, active as SERMs.

According to one embodiment, the present invention provides pharmaceutical composition comprising as an active ingredient an isoflavone derivative having the general formula (I): 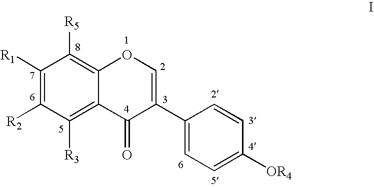
wherein
R₁ is selected from the group consisting of OH, OCH₃ OGlc and OR′COOX;
R₂ is selected from the group consisting of H and R′COOX;
R₃ is selected from the group consisting of H, OH, R′COOX and OR′COOX;
R₄ is selected from the group consisting of H, CH₃ and R′COOX;
R₅ is selected from the group consisting of H and R′COOX;
R′ is selected from the group consisting of (C₁-C₆)alkyl, (C₁-C₂₀)alkoxy, (C₁-C₂₀) alkenyl;
X is selected from the group consisting of H and (CH₂)n-Y wherein Y is CH₃ or NH₂ and n=0-10;
   with the proviso that at least one of R₁, R₂, R₃, R₄ or R₅ comprises carboxy group; further comprising a pharmaceutically acceptable diluent or carrier.

As used herein, a “pharmaceutical composition” refers to a preparation with one or more of the compounds described herein, or physiologically acceptable salts thereof, together with other chemicals components such as physiological acceptable diluents or carriers. The purpose of a pharmaceutical composition is to facilitate administration of a compound to an organism.

Pharmaceutical composition of the present invention may be manufactured by processes well known in the art, e.g. by means of conventional mixing, dissolving, granulating, grinding, pulverizing, dragee-making, levigating, emulsifying, encapsulating, entrapping or lyophilizing processes.

Pharmaceutical composition for use in accordance with the present invention thus may be formulated in conventional manner using one or more acceptable diluents or carriers comprising excipients and auxiliaries, which facilitate processing of the active compounds into preparations, which can be used pharmaceutically. Proper formulation is dependent on the route of administration chosen.

More particularly the present invention relates to pharmaceutical compositions for parenteral and oral administration.

Pharmaceutical compositions for parenteral administration are formulated for intravenous injections, intravenous infusion, intradermal, intralesional, intramuscular, and subcutaneous injections or depots; or they may be administered parenterally by means other than injection, for example, they could be introduced laparascopically, intravesicularly, or via any orifice not related to the gastrointestinal tract.

For oral administration, the compound can be formulated readily by combining the active compounds with pharmaceutically acceptable diluents or carriers well known in the art. Such carriers enable the compounds of the invention to be formulated as capsules, dragees, pills, tablets, gels, liquids, slurries, suspensions, syrups and the like, for oral ingestion by a patient.

According to another aspect, the present invention is related to a method for treating estrogen-related conditions. Such conditions generally include (but are not limited to) obesity, breast cancer, osteoporosis, endometriosis, cardiovascular disease, prostate cancer, menopausal syndromes, hair loss (alopecia), type-II diabetes, Alzheimer's disease, urinary incontinence, GI tract conditions, spermatogenesis, vascular protection after injury, restenosis, learning and memory, CNS effects, plasma lipid levels, acne, cataracts, hirsutism, other solid cancers (such as colon, lung, ovarian, melanoma, CNS, and renal), multiple myeloma, and lymphoma.

According to one embodiment the present invention relates to a method comprising the step of administering to a subject in need thereof a therapeutically effective amount of an isoflavone derivative as an estrogen receptor modulator.

According to one currently preferred embodiment, the isoflavone derivative is selected from the group consisting of 6-carboxymethyl biochanin A, 8-carboxymethyl biochanin A, 7-(O)-carboxymethyl daidzein, 7-(O)-carboxymethyl formononetin and 6-carboxymethyl genistein.

According to another aspect the present invention relates to affinity targeting of isoflavone conjugates to normal and malignant cells expressing ER, the presence of the carboxy group in the isoflavone derivatives permitting the synthesis of isoflavone conjugates.

According to one embodiment, the present invention provides isoflavone conjugates having the general formula (II): 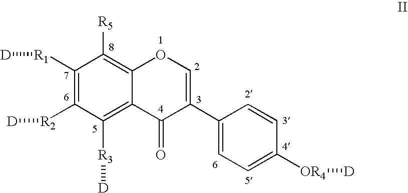
wherein
R₁ is selected from the group consisting of OH, OCH₃ OGlc, OR′COOX and OR′CO;
R₂ is selected from the group consisting of H, R′COOX and R′CO;
R₃ is selected from the group consisting of H, OH, R′COOX, R′CO, OR′COOX and OR′CO;
R₄ is selected from the group consisting of H, CH₃, R′COOX and R′CO;
R₅ is selected from the group consisting of H, R′COOX and R′CO;
D may be absent or is a bioactive moiety;
R′ is selected from the group consisting of (C₁-C₆)alkyl, (C₁-C₂₀)alkoxy, (C₁-C₂₀) alkenyl;
X is selected from the group consisting of H and (CH₂)n-Y wherein Y is CH₃ or NH₂ and n=0-10;
   with the proviso that at least one of R₁, R₂, R₃, R₄ or R₅ is conjugated to D.

According to one embodiment, D is selected from the group consisting of a cytotoxic compound, a cytostatic compound, an antisense compound, an anti-viral agent, a specific antibody, a biodegradable carrier and an imaging and detection agents other than Keyhole Limpet Hemocyanin (KLH), ovalbumin and Horseradish peroxidase (HRP).

According to another embodiment, D is a cytotoxic compound selected from, but not restricted to: agents inhibitory of DNA synthesis and function: adriamycin, bleomycin, chlorambucil, cisplatin, daunomycin, ifosfamide and melphalan; agent inhibitory of microtubule (mitotic spindle) formation and function: vinblastine, vincristine, vinorelbine, paclitaxel (taxol) and docetaxel; anti metabolites: cytarabine, fluorouracil, fluroximidine, mercaptopurine, methotorexate, gemcitabin and thioquanine; alkylating agents: mechlorethamine, chlorambucil, cyclophosphamide, melphalan and methotrexate; antibiotics: bleomycin and mitomycin; nitrosoureas: carmustine (BCNU) and lomustine; inorganic ions: carboplatin, oxaloplatin; interferon and asparaginase; hormones: tamoxifen, leuprolide, flutamide and megestrol acetate.

According to one preferred embodiment, the cytotoxic substance D is an anti-tumor agent.

According to one currently preferred embodiment the anti-tumor agent is daunomycin, and the carboxy-isoflavone is selected from the group consisting of 6-carboxymethyl biochanin A, 8-carboxymethyl biochanin A, 7-(O)-carboxymethyl daidzein, 7-(O)-carboxymethyl formononetin and 6-carboxymethyl genistein.

According to another preferred embodiments, D is an imaging compound selected from, but not restricted to paramagnetic particles: gadolinium, yttrium, lutetium and gallinum; radioactive moieties: radioactive indium, rhenium and technetium fluorescent dyes: fluorescein isothiocyanate (FITC), green fluorescent protein (GFP), Cyan fluorescent protein (CFP), rhodamine I, II, III and IV, rhodamine B and rosamine.

According to another aspect of the embodiment, a plurality of bioactive moieties (D) is conjugated to at least two of R₁, R₂, R₃, R₄ or R₅, wherein D may be the same or different at each occurrence.

Alternatively and preferably, a plurality of bioactive moieties D are conjugated to at least two of R₁, R₂, R₃, R₄ or R₅, wherein at least one D is a therapeutic agent and at least one D is a biodegradable carrier. In this more preferred embodiment, at least one D is a polyvalent natural or synthetic peptide or polypeptide, having free carboxy or amino groups.

The present invention further discloses a method for site directed chemotherapy, using the cytotoxic isoflavone conjugate for affinity drug targeting to an estrogen receptor, preferably to estrogen receptor subtype β.

Current cancer therapy involves the use of antimitotic drugs exemplified by adriamycin, vincristine, cisplatin, methotrexate and daunomycin, all with undesirable side effects on normal cells. The present invention now discloses cytotoxic isoflavone conjugates for site directed or targeted chemotherapy.

According to one currently preferred embodiments, the cytotoxic isoflavone conjugates are selected from the group of 6-carboxymethyl biochanin A-daunomycin, 8-carboxymethyl biochanin A-daunomycin, 7-(O)-carboxymethyl daidzein-daunomycin, 7-(O)-carboxymethyl formononetin-daunomycin and 6-carboxymethyl genistein-daunomycin, showing about 10 to 130 fold more toxicity towards cells expressing mainly ERβ (e.g. R1, VSMC, NCI-H295R and colo320) compared to free daunomycin. Surprisingly, 6-Carboxymethyl biochanin A-daunomycin also shows potent cytotoxic activity towards E304 cell, bearing mainly ERα. No cytotoxic activity was shown for normal rat enterocytes (IEC) cells devoid of ER when treated with 6-Carboxymethyl genistein-daunomycin.

According to yet another embodiment the present invention relates to a method for site directed chemotherapy using an isoflavone conjugate containing a cytotoxic agent with or without a biodegradable carrier for affinity drug targeting to an estrogen receptor, preferably estrogen receptor subtype β.

According to yet another aspect the present invention discloses a method for site directed diagnosis, using the labeled isoflavone conjugate for affinity label targeting to an estrogen receptor, preferably to estrogen receptor subtype β.

According to one currently preferred embodiment the labeling is exemplified by, but not limited to magnetic particles, radioactive moieties or fluorescent dyes.

According to another aspect, the present invention relates to a pharmaceutical composition comprising as an active ingredient an isoflavone conjugate.

According to one embodiment, the present invention provides pharmaceutical composition comprising as an active ingredient an isoflavone conjugate having the general formula II: 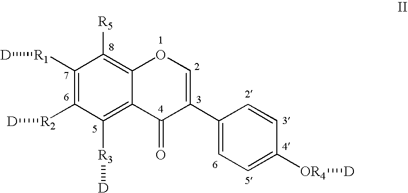
wherein
R₁ is selected from the group consisting of OH, OCH₃ OGlc, OR′COOX and OR′CO;
R₂ is selected from the group consisting of H, R′COOX and R′CO;
R₃ is selected from the group consisting of H, OH, R′COOX, R′CO, OR′COOX and OR′CO;
R₄ is selected from the group consisting of H, CH₃, R′COOX and R′CO;
R₅ is selected from the group consisting of H, R′COOX and R′CO;
D may be absent or is a bioactive moiety;
R′ is selected from the group consisting of (C₁-C₆)alkyl, (C₁-C₂₀)alkoxy, (C₁-C₂₀) alkenyl;
X is selected from the group consisting of H and (CH₂)n-Y wherein Y is CH₃ or NH₂ and n=0-10;
   with the proviso that at least one of R₁, R₂, R₃, R₄ or R₅ is conjugated to D, further comprising pharmaceutically acceptable diluent or carrier.

According to one embodiment the present invention relates to pharmaceutical compositions of isoflavone conjugates for parenteral and oral administration.

According to one embodiment, pharmaceutical compositions for parenteral administration are formulated for intravenous injections, intravenous infusion, intradermal, intralesional, intramuscular, and subcutaneous injections or depots; or they may be administered parenterally by means other than injection, for example, they could be introduced laparascopically, intravesicularly, or via any orifice not related to the gastrointestinal tract. For oral administration, the compound can be formulated readily by combining the active compounds with pharmaceutically acceptable diluents or carriers well known in the art. Such carriers enable the compounds of the invention to be formulated as tablets, pills, dragees, capsules, liquids, gels, syrups, slurries, suspensions, and the like, for oral ingestion by a patient.

According to another aspect the present invention relates to a method comprising the step of administering to a subject in need thereof a therapeutically effective amount of a pharmaceutical composition comprising as an active ingredient a cytotoxic isoflavone conjugate.

According to one another aspect the present invention relates to a method comprising the step of administering to a subject in need thereof a diagnostically effective amount of pharmaceutical composition comprising as an active ingredient a labeled isoflavone conjugate.

The principles of the invention, using carboxy derivatives of isoflavones as active selective estrogen receptor modulators, and their conjugates with a bioactive moiety for selective delivery to cells that carry estrogen receptor (ER), according to the present invention, may be better understood with reference to the following non-limiting examples.

### EXAMPLES

### Example 1

### Preparation of 6-carboxymethyl Biochanin A and 8-carboxymethyl Biochanin A

The synthesis of 6-carboxymethyl genistein and that of 7-(O)-carboxymethyl daidzein has been reported previously (Kohen F. et al. 1999 A nonisotopic enzyme-based immunoassay for assessing human exposure to genistein. Nutr. Cancer 35:96-103; Kohen F. et al. 1998 The measurement of the isoflavone daidzein by time resolved fluorescent immunoassay: a method for assessment of dietary soya exposure. J. Steroid Biochem. Mol. Biol. 64:217-222). The present example describes the preparation of the novel carboxy derivative of biochanin A, 6-carboxymethyl biochanin A and 8-carboxymethyl biochanin A.

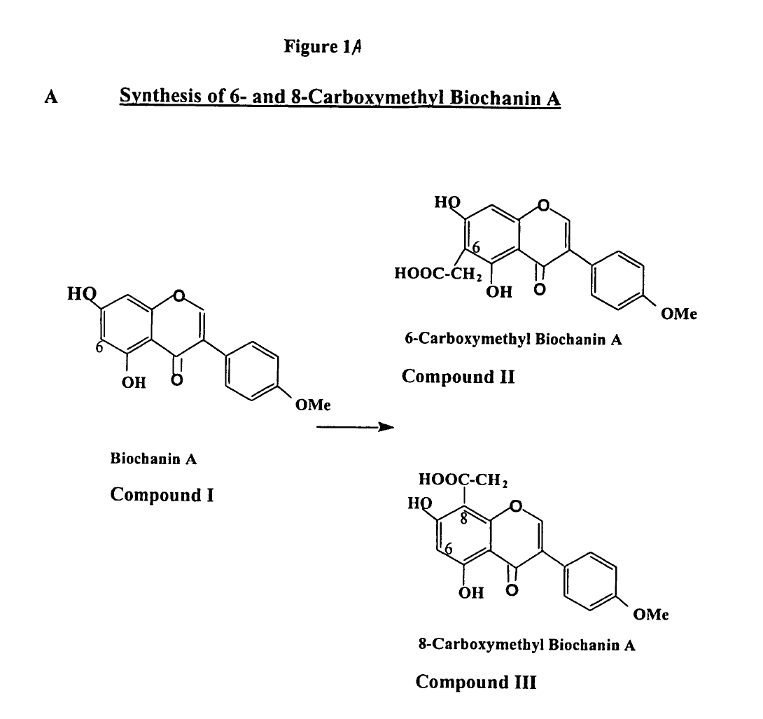

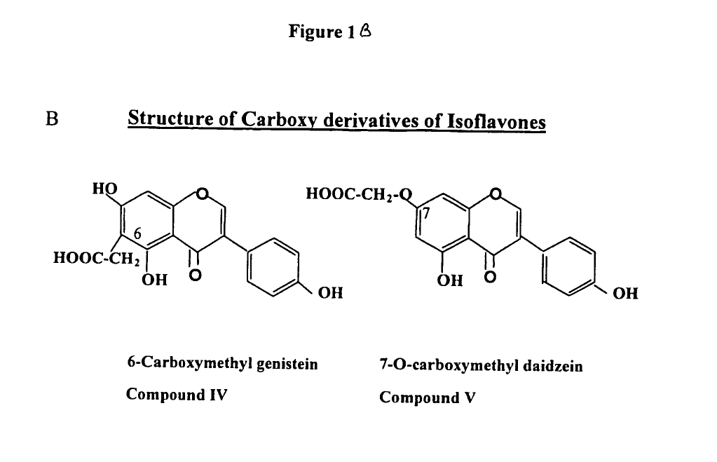

Sodium (0.31 g), cut into small pieces, was added under nitrogen to a 3-necked flask containing n-propanol (8 ml). After dissolution of sodium, biochanin A (100 mg) (compound 1, FIG. 1A) in 6 ml of n-propanol was added. The reaction mixture was stirred for 15 min and the bromoacetic acid (0.377 g) in 2 ml of n-propanol was added. A precipitate was formed immediately, and the color of the reaction changed gradually from yellow to green. The reaction mixture was stirred for 2 h at 60° C. After cooling to room temperature, water was added, and the solvent was evaporated. The residue was acidified with 5N HCl to pH 3 and extracted with ether. The organic phase was washed with water, separated, dried with anhydrous magnesium sulphate, evaporated and chromatographed on Silica gel 60. Elution of Silica gel 60 with methanol:chloroform:acetic acid (5:94.7:0.3) yielded the desired mono-addition product (20 mg) with an R_{f} of 0.46-0.5 in the solvent system chloroform:methanol:acetic acid (89.7:10:0.3) while biochanin A showed an R_{f} of 0.8. The ¹H NMR spectrum of the carboxy derivatives of biochanin A (compound II and III, FIG. 1A) in deuterated dimethyl sulfoxide showed the following signals: δ: 8.3 (1H, 2-H), 7.46 (2H, d, J=2 Hz, 2′H and 6′H), 6.97 (2H, d, J=2 Hz, 3′H and 5′H), 6.28 (1H, s, 8-H) for 6-carboxymethyl biochanin A and 6.44 (1H, s, 6-H) for 8-carboxymethyl biochanin A, 3.6 (2H, s, —CH₂—COOH) and 3.74 (3H, s, OMe). The most characteristic signals in the NMR spectrum of the carboxymethyl derivatives of biochanin A were a singlet at δ 6.28, which can be attributed to 8-H (6-carboxymethyl biochanin A), a singlet at δ 6.44, which can be attributed to 6-H (8-carboxymethyl biochanin A), and a singlet at δ 3.6 equivalent to 2H, attributed to the methylene group in —CH₂COOH. In addition, when 6-carboxymethyl biochanin A was synthesized, the NMR spectrum of this carboxymethyl derivative of biochanin A did not have a signal for 6-H, which is expected to be a doublet at δ 6.33 characteristic of genistein and biochanin A. These data indicate that the carboxymethyl group was attached to the 6-position of biochanin A. When 8-carboxymethyl biochanin A was synthesized, the NMR spectrum of this carboxymethyl derivative of biochanin A did not have a signal for 8-H, which is expected to be a doublet at δ 6.20 characteristic of biochanin A. These data indicate that the carboxymethyl group was attached to the 8-position of biochanin A.

### Example 2

### Synthesis of Isoflavone Daunomycin Conjugates

The carboxy derivatives of isoflavones were coupled to the cytotoxic drug daunomycin in a two-steps procedure. In the first step of the reaction, the carboxy derivative of isoflavones was treated with N-hydroxysuccinimide and carbodiimide to form an active ester. In the second step of the reaction the activated ester reacted at pH 8 with the amino group of the sugar part of daunomycin to form the cytotoxic isoflavone conjugates.

As an example the preparation of 6-carboxymethyl genistein daunomycin conjugate is described herein.

6-carboxymethyl genistein (compound IV, FIG. 1B) (3.76 mg) was dissolved in dry dioxane (366 μl). N-hydroxysuccinimide (2.2 mg) and carbodiimide (2.9 mg) were then added, and the reaction mixture was left overnight at room temperature. The reaction mixture was then analyzed by thin layer chromatography using CHCl₃: MeOH: Acetic acid (84.75:15:0.25) as the developing solvent, and an R_{f} of 0.95 was obtained, indicating that the active ester of 6-carboxymethyl genistein was formed. In the same solvent system 6-carboxymethyl genistein showed an R_{f} of 0.4.

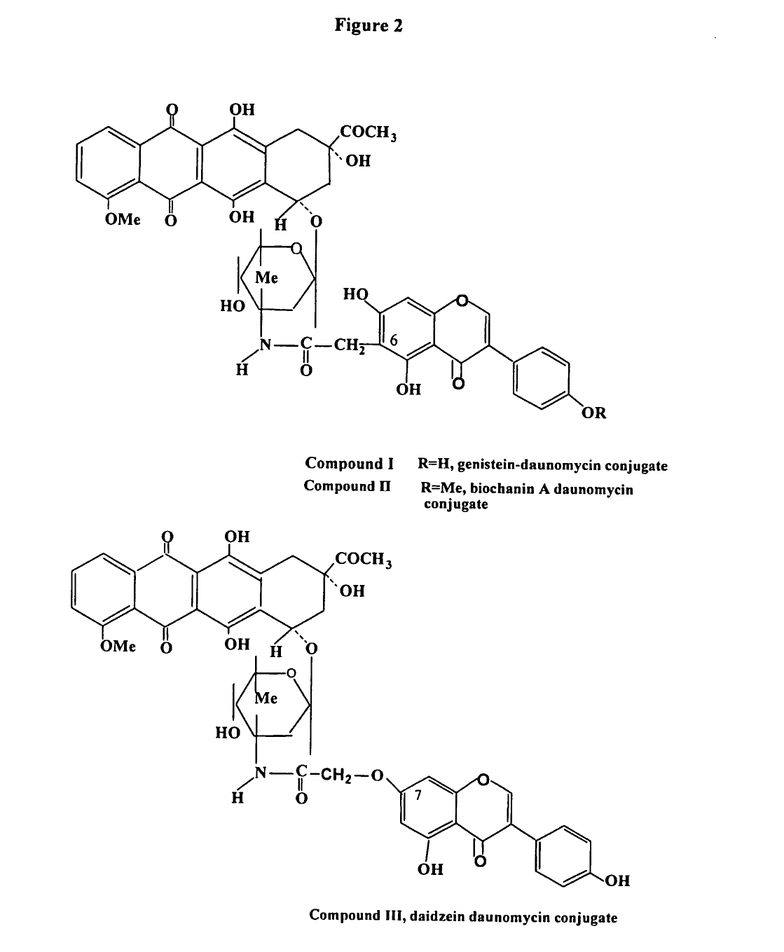

Daunomycin (0.8 mg) was dissolved in 20 μl of 0.13 M NaHCO₃. A portion of the active ester prepared above (110 μl) was then added drop wise, and the reaction mixture was stirred overnight at 4° C. The pH of the reaction mixture was subsequently adjusted to 8. The desired product, 6-carboxymethyl-genistein daunomycin conjugate (compound I, FIG. 2), was isolated by ethyl acetate extraction of the reaction mixture. The organic phase was then separated from the aqueous phase, dried with magnesium sulfate and evaporated. The concentration of the conjugate was then determined at 495 nm using an absorption coefficient (ε) of 10000. The electron spray (ES+) mass spectrum of 6-carboxymethyl genistein daunomycin conjugate gave the expected molecular weight of 859.90, corresponding to C₄₄H₃₉NO₁₆Na.

### Example 3

### Estrogen Receptor-Binding Assays

Recombinant ERα or ERβ protein (12 pmol/ml) in 10 μl of binding buffer (10 mM Tris, pH 7.5, containing 10% glycerol, 2 mM dithiothreitol (DTT), and 1 mg/ml BSA) was incubated in streptavidin-coated microtiter plates for 30 min at room temperature, in the absence or presence of serial dilutions of 17β-estradiol in 50 μl of binding buffer or of the compounds to be tested. [3H]-17β-estradiol (3 nM) in 50 μl of binding buffer was added to each well and the mixtures were incubated overnight at 4° C. Biotinylated anti-ER antibody (α or β, prepared as described in Strasburger C J & Kohen F 1990 Methods Enzymol. 184:481-496), 100 ng/well in 100 μl of binding buffer, was added to each well, and the reaction mixtures were incubated with shaking for 2 h and 30 min at room temperature. The reaction mixtures were then decanted, and each well was washed once with binding buffer. Dilute sodium hydroxide (0.1 N, 300 μl) was added to each well. After shaking for 20 min, an aliquot (200 μl) was removed from each well and added to a vial containing scintillation fluid. The vials were then counted for radioactivity in a beta scintillation counter.

The binding assays showed that genistein and 6-carboxy genistein inhibit the binding of [3]H-estradiol to ERβ with relative binding affinity values (IC50) of 1 μM and 0.2 μM respectively. On the other hand genistein inhibits the binding of [3H]-estradiol to ERα with an IC50 of 0.1 μM while 6-carboxymethyl genistein did not significantly inhibit the binding of [3H]-estradiol to ERα (IC50<0.01). Daidzein, 7-(O)-carboxymethyl daidzein, biochanin A, and 6-carboxymethyl biochanin A did not show any significant binding activity either to ERα or ERβ. Under the same experimental conditions the IC50 of estradiol to ERα is 0.8 nM and to ERβ is 1 nM.

### Example 4

### Stimulation of the Specific Activity of CK by Biochanin A Analogs In Vivo

Immature (25 days old Wistar derived) female rats were injected with E2 (5 μg/rat), biochanin A (0.5 mg/rat), 6-carboxymethyl biochanin A (250 μg/rat or 0.5 mg/rat) or with the combination of estradiol+biochanin A or estradiol+6-carboxymethyl biochanin A. Rats were injected intraperitoneally (i.p.), with 0.05% ethanol in PBS serving as a control. The rats were killed by decapitation 24 h after i.p. injection. The various organs were removed and stored at −20° C. until processed for CK activity as previously described (Somjen D. et al. 1998 Hypertension 32:39-45).

Estradiol and biochanin A stimulated the CK specific activity in all the rat tissues that were examined (uterus, pituitary, epiphysis, diaphysis, aorta, and left ventricle of the heart, Table 1) while 6-carboxymethyl biochanin A increased the CK specific activity in all the rat tissues with the exception of the uterus. The stimulatory response of E2 to CK specific activity was inhibited in all the tissues when rats were treated with a combination of E2 plus 6-carboxymethyl biochanin A, showing that 6-carboxymethyl biochanin A acts like an SERM in these tissues (Table 1). It seems probable that the introduction of a carboxy group to genistein and to biochanin A at position 6 of the molecule imparts anti-estrogenic properties to these isoflavones.

TABLE 1
Stimulation of the specific activity of creatine kinase (CK)
by estrogen and isoflavone derivatives in rat tissues in vivo,
presented as CK specific activity (experimental/control)

| | | | | | | 6-carboxy- |
|---|---|---|---|---|---|---|
| | | | | 6-carboxy- | | methyl- |
| | | | | methyl- | Biochanin A + | biochanin A + |
| | Control | Estradiol | Biochanin A | biochanin A | Estradiol | Estradiol |
| Epi | 1.1 ± 0.09 | 1.85 ± 0.16** | 2.38 ± 0.18** | 1.61 ± 0.17* | 2.09 ± 0.19** | 1.02 ± 0.29 |
| Dia | 1 ± 0.16 | 2.75 ± 0.23** | 1.9 ± 0.23** | 1.51 ± 0.05* | 2.78 ± 0.13** | 0.84 ± 0.07 |
| Ut | 1 ± 0.11 | 1.49 ± 0.13* | 1.42 ± 0.13* | 0.89 ± 0.12 | 1.48 ± 0.11* | 1.02 ± 0.22 |
| Ao | 1 ± 0.1 | 2.43 ± 0.06** | 2 ± 0.18** | 1.63 ± 0.11* | 2.38 ± 0.06** | 1.33 ± 0.15 |
| LV | 1 ± 0.09 | 1.53 ± 0.13* | 1.42 ± 0.04* | 1.91 ± 0.16** | 1.6 ± 0.09* | 1.1 ± 0.12 |
| Pi | 1 ± 0.14 | 1.45 ± 0.05* | 1.58 ± 0.05* | 1.54 ± 0.05* | 1.66 ± 0.14* | 1.16 ± 0.08 |

The results are expressed as means ± SD for n = 5 and further expressed as experimental over control where the control is given a value of 1.0.
*p < 0.05;
**p < 0.01;
treated vs. control;
Abbreviations used:
Epi: epiphysis;
Di: diaphysis;
Ut: uterus;
Ao: aorta;
LV: left ventricle of the heart;
Pi: pituitary

### Example 5

### Cytotoxicity Studies of Isoflavone-Daunomycin Conjugates in Cultured Cells

In the first phase of the study the ability of the carboxymethyl derivatives of the isoflavones to stimulate DNA synthesis in vitro was studied in normal and malignant cells. Cells cultures used were as follows:

a. Human Umbilical Artery Smooth Muscle Cells (VSMC):

Human umbilical artery smooth muscle cells, expressing mainly ERβ, were prepared as previously described with minor modifications (Somjen D. et al. 1998 Hypertension 32:39-45). In brief, umbilical cords were collected shortly after delivery. The umbilical arteries were isolated by dissection, cleaned of blood and adventitia and then cut into tiny slices (1-3 mm). The segments were kept in culture in medium 199 containing 20% FCS, glutamine and antibiotics. Cell migration was detected within 5-7 days. Cells were fed twice a week and, upon confluence, trypsinized and transferred to 24-well dishes. Cells were used only at passages 1-3 when expression of smooth muscle actin was clearly demonstrable.

b. Endothelial Cells (E304):

E304 cells, expressing mainly ERα, an endothelial cell line derived from a human umbilical vein, were purchased from American Type Culture Collection (ATCC), Rockville, Md., and grown in medium 199 containing 10% FCS, glutamine and antibiotics.

c. Rat Enterocytes; IEC and R1 Cells:

Cell lines were obtained from Prof. N. Arber, Ichilov Hospital, Tel-Aviv, Israel and grown as described previously (Arber N et al. 1996 Oncogene 12:1903-1908).

d. Human Adrenocortical Carcinoma Cells (NCI-H295R):

These cells were purchased from ATTC (Rockville, Md.) and grown in Dulbecco's modified Eagle's medium containing antibiotics.

e. Human Colon Cancer Cells (colo 320)

These cells were purchased from ATTC (Rockville, Md.) and grown in RPMI medium containing 20 mM HEPES and 10% fetal calf serum.

Assessment of DNA synthesis was performed by [³H]-thymidine incorporation in these cells. Cells were grown until subconfluence and then treated with various hormones or agents as indicated. Forty-eight hours later, [³H] thymidine was added for two hours. Cells were then treated with 10% ice-cold trichloroacetic (TCA) for 5 min and washed twice with 5% TCA and then with cold ethanol. The cellular layer was dissolved in 0.3 ml of 0.3M NaOH, samples were taken and [³H] thymidine incorporation into DNA was determined. The concentration of hormone to produce half-maximal induction (EC₅₀) or inhibition (IC₅₀) was calculated from the dose response curves.

All three carboxymethyl derivatives of the isoflavones increased DNA synthesis in these cells with EC₅₀ ranging from 2 nM to 200 nM. In the second phase the cytotoxicity of isoflavone-daunomycin conjugates was tested after 48 h of incubation in normal cells [VSMC, E304, non transformed enterocytes (TEC)] and malignant cells [human adrenocortical carcinoma (NCI-H295R), human colon cancer cells (colo320) and c-K-ras transformed rat enterocytes (R1)] using uptake of [³H]-thymidine as a proliferation marker. In cells expressing mainly ERβ, the IC₅₀ of 6-carboxymethyl genistein daunomycin conjugate (compound I, FIG. 2) for inhibition DNA synthesis was 20 nM in VSMC, 18 nM in NCI-H295R and 70 nM in R1 cells. Under the same experimental conditions the IC₅₀ of daunomycin was 700 nM in VSMC, 800 nM in NCI-295R and 850 nM in R1 cells.

The 7-(O)-carboxymethyl daidzein-daunomycin conjugate (compound III, FIG. 2) exhibited the same sort of cytotoxicity as the cytotoxic genistein derivative with an IC₅₀ of 22 nM in VSMC cells and 7 nM in NCI-H295R cells.

Similarly, 6-carboxymethyl biochanin A daunomycin conjugate (Compound II, FIG. 2) was more toxic than daunomycin in colon cancer cells (colo320) and NCI-H295R cells with IC₅₀ of 40 nM and 60 nm respectively.

On the other hand in E304 cells expressing mainly ERα, the IC₅₀ of 6-carboxymethyl genistein daunomycin conjugate was 60 nM and in non-transformed enterocytes IEC the IC₅₀ was 2000 nM. Interestingly, the IC₅₀ of 6-carboxymethyl biochanin A daunomycin conjugate was 5 nM in E304 endothelial cells. Under the same experimental conditions, the IC₅₀ of daunomycin in E304 cells was 300 nM.

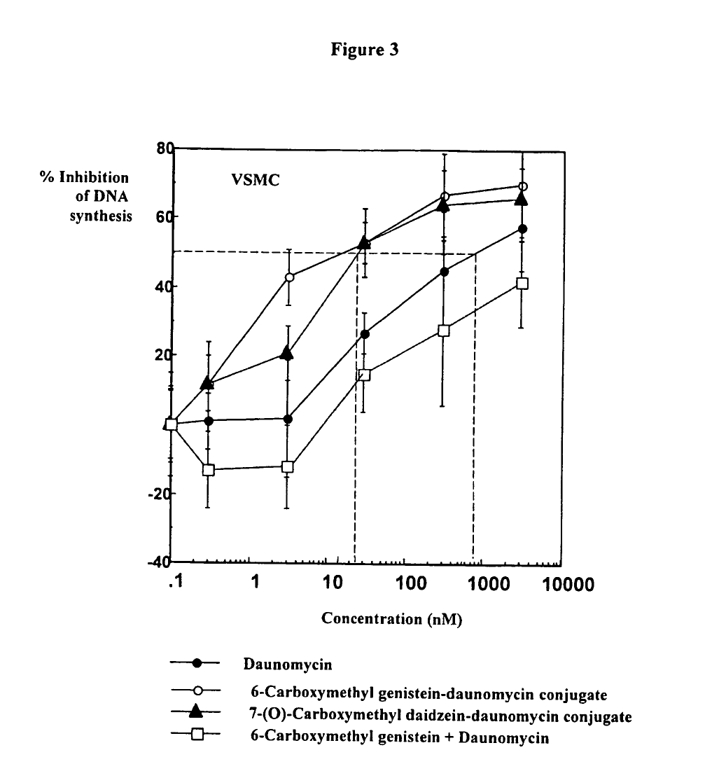

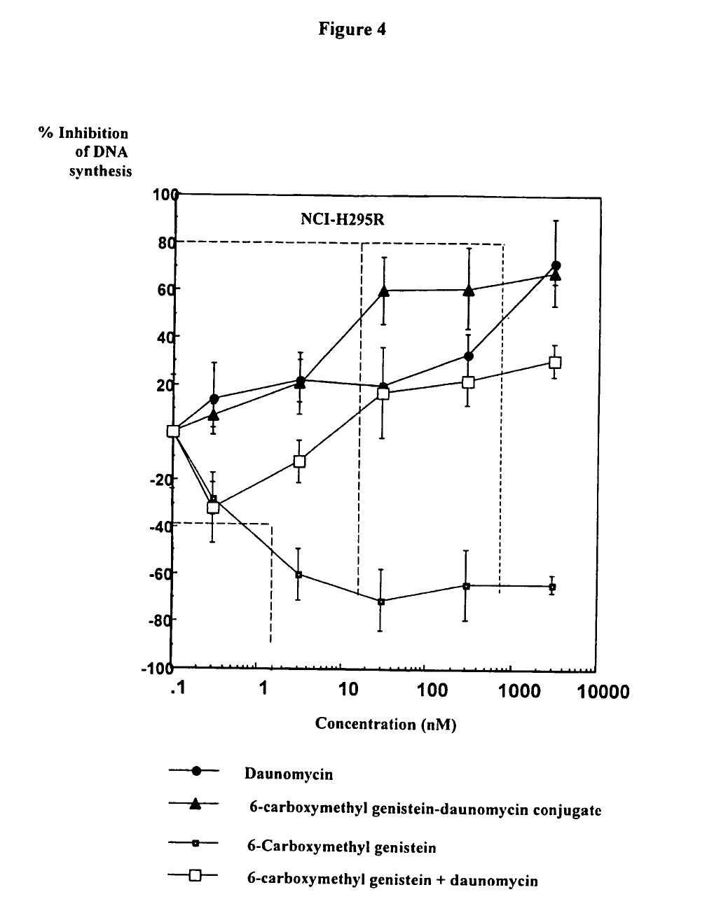

Moreover, when VSMC and NCI-295R cells were treated with a combination of carboxymethyl genistein and daunomycin the observed IC₅₀ was >3000 nM, indicating that the cytotoxicity of the isoflavone-daunomycin conjugates was receptor mediated. On the other hand, in these cells 6-carboxymethyl genistein induced proliferation with EC₅₀ of 3 nM in VSMC and 2 nM in NCI-H295R cells (see FIG. 4). FIGS. 3 and 4 show the dose dependent reduction in cell proliferation of VSMC and NCI-H295R cells upon treatment with these cytotoxic conjugates, and Table 2 shows the potency of these isoflavone cytotoxic conjugates in terms of cytotoxicity in all the cultured cells.

TABLE 2
Potency of isoflavone daunomycin conjugates determined by
in vitro inhibition of DNA synthesis, presented as the concentration
(nM) required for 50% inhibition of DNA synthesis (IC₅₀).

| | Cell type | | | | | |
|---|---|---|---|---|---|---|
| Addition to cells | E304 | VSMC | NCI-H295R | R1 | IEC | Colo320 |
| Daunomycin | 800 | 650 | 800 | 850 | 550 | 300 |
| Cbio-daunomycin | 5# | ND | 60# | ND | ND | 40 |
| Cgen-daunomycin | 60 | 12 | 16 | 70 | 2000 | ND |
| Cdaid-daunomycin | ND | 22 | 6 | ND | ND | ND |
| Cgen + Daunomycin | ND | >3000 | >3000 | ND | ND | ND |

Abbreviations used:
E304 = endothelial cells;
VSMC = human vascular smooth muscle cells;
NCI-H295R = human adrenocortical carcinoma cells;
R1 = c-K-ras transformed rat enterocytes;
IEC = nontransformed rat enterocytes;
Colo320 = human colon cancer cell lines;
Cgen = 6-carboxymethyl genistein;
Cdaid = 7-(O)-carboxymethyl daidzein;
Cbio: 6-carboxymethl biochanin A.
ND = not determined
#In this experiment the IC₅₀ for daunomycin was 300 nM.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the scope of the invention, which is defined in the claims which follow.

1. An estrogen binding isoflavone derivative having the general formula (I): 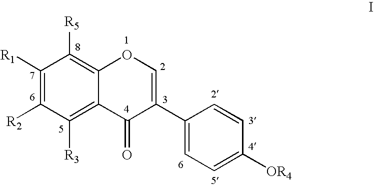
wherein
R₁ is selected from the group consisting of OH, OCH₃ OGlc and O—R′COOX;
R₂ is selected from the group consisting of H and R′COOX;
R₃ is selected from the group consisting of H, OH, R′COOX and OR′COOX;
R₄ is selected from the group consisting of H, CH₃ and R′COOX;
R₅ is selected from the group consisting of H and R′COOX;
R′ is selected from the group consisting of (C₁-C₆)alkyl, (C₁-C₂₀)alkoxy, (C₁-C₂₀)alkenyl;
X is selected from the group consisting of H and (CH₂)n-Y wherein Y is CH₃ or NH₂ and n=0-10;
with the proviso that at least one of R₁, R₂, R₃, R₄ or R₅ comprises carboxy group.

2. The isoflavone derivative of claim 1 wherein the isoflavone is selected from the group consisting of diadzein, genistein, formononetin and biochanin A.

3. The isoflavone derivative of claim 1 wherein the isoflavone is biochanin A.

4. The isoflavone derivative of claim 1 wherein the isoflavone is formononetin.

5. The isoflavone derivative of claim 1 selected for the group consisting of 7-(O)-carboxymethyl daidzein, 6-carboxymethyl genistein, 6-carboxymethyl biochanin A, 8-carboxymethyl biochanin A and 7-(O)-carboxymethyl formononetin.

6. The isoflavone derivative of claim 1 wherein the derivative is 6-carboxymethyl biochanin A.

7. The isoflavone derivative of claim 1 wherein the derivative is 8-carboxymethyl biochanin A.

8. The isoflavone derivative of claim 1 wherein the derivative is 7-(O)-carboxymethyl formononetin.

9. A pharmaceutical composition comprising as an active ingredient an estrogen receptor binding isoflavone derivative having the general formula (I): 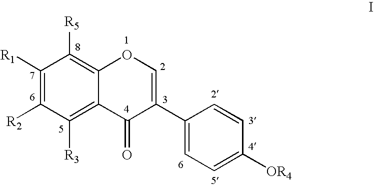
wherein
R₁ is selected from the group consisting of OH, OCH₃ OGlc and OR′COOX;
R₂ is selected from the group consisting of H and R′COOX;
R₃ is selected from the group consisting of H, OH, R′COOX and OR′COOX;
R₄ is selected from the group consisting of H, CH₃ and R′COOX;
R₅ is selected from the group consisting of H and R′COOX;
R′ is selected from the group consisting of (C₁-C₆)alkyl, (C₁-C₂₀)alkoxy, (C₁-C₂₀)alkenyl;
X is selected from the group consisting of H and (CH₂)n-Y wherein Y is CH₃ or NH₂ and n=0-10;
with the proviso that at least one of R₁, R₂, R₃, R₄ or R₅ comprises carboxy group, further comprising a pharmaceutically acceptable diluent or carrier.

10. The pharmaceutical composition of claim 9 wherein said isoflavone derivative is selected from the group consisting of 6-carboxymethyl biochanin A, 8-carboxymethyl biochanin A, 7-(O)-carboxymethyl daidzein, 7-(O)-carboxymethyl formononetin and 6-carboxymethyl genistein.

11. The pharmaceutical composition of claim 9 wherein said isoflavone derivative is 6-carboxymethyl biochanin A.

12. The pharmaceutical composition of claim 9 wherein said isoflavone derivative is 8-carboxymethyl biochanin A.

13. The pharmaceutical composition of claim 9 wherein said isoflavone derivative is 7-(O)-carboxymethyl formononetin.

14. The pharmaceutical composition of claim 9 wherein the formulation is selected from dosage forms suitable for parenteral or oral administration.

15. The pharmaceutical composition of claims 14 wherein the parenteral formulation is selected from a group consisting of forms suitable for intravenous injections, intravenous infusion, intradermal, intralesional, intramuscular and subcutaneous injections or depots, or for administering laparascopically and intravesicularly.

16. The pharmaceutical composition of claims 14 wherein the formulation of oral administration is selected from liquids, suspensions, slurries, syrups, gels, tablets, pills, dragees and capsules.

17. A method for treating a subject in need thereof comprising the step of administering to said subject a therapeutically effective amount of estrogen receptor modulating isoflavone derivative, having the general formula I: 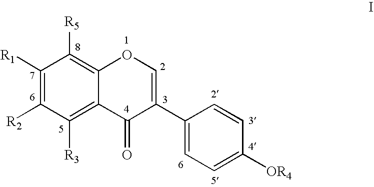
wherein
R₁ is selected from the group consisting of OH, OCH₃ OGlc and OR′COOX;
R₂ is selected from the group consisting of H and R′COOX;
R₃ is selected from the group consisting of H, OH, R′COOX and OR′COOX;
R₄ is selected from the group consisting of H, CH₃ and R′COOX;
R₅ is selected from the group consisting of H and R′COOX;
R′ is selected from the group consisting of (C₁-C₆)alkyl, (C₁-C₂₀)alkoxy, (C₁-C₂₀)alkenyl;
X is selected from the group consisting of H and (CH₂)n-Y wherein Y is CH₃ or NH₂ and n=0-10;
with the proviso that at least one of R₁, R₂, R₃, R₄ or R₅ comprises carboxy group.

18. The method of claim 17 wherein said estrogen receptor modulating isoflavone derivative is selected from the group consisting of 6-carboxymethyl biochanin A, 8-carboxymethyl biochanin A, 7-(O)-carboxymethyl daidzein, 7-(O)-carboxymethyl formononetin and 6-carboxymethyl genistein.

19. The method of claim 17 wherein said estrogen receptor modulating isoflavone derivative is 6-carboxymethyl biochanin A.

20. The method of claim 17 wherein said estrogen receptor modulating isoflavone derivative is 8-carboxymethyl biochanin A.

21. The method of claim 17 wherein said estrogen receptor modulating isoflavone derivative is 7-(O)-carboxymethyl formononetin.

22. An isoflavone conjugate having the general formula (II 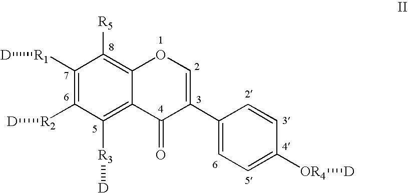
wherein
R₁ is selected from the group consisting of OH, OCH₃ OGlc, OR′COOX and OR′CO;
R₂ is selected from the group consisting of H, R′COOX and R′CO;
R₃ is selected from the group consisting of H, OH, R′COOX, R′CO, OR′COOX and OR′CO;
R₄ is selected from the group consisting of H, CH₃, R′COOX and R′CO;
R₅ is selected from the group consisting of H, R′COOX and R′CO;
D may be absent or is a bioactive moiety;
R′ is selected from the group consisting of (C₁-C₆)alkyl, (C₁-C₂₀)alkoxy, (C₁-C₂₀)alkenyl;
X is selected from the group consisting of H and (CH₂)n-Y wherein Y is CH₃ or NH₂ and n=0-10;
with the proviso that at least one of R₁, R₂, R₃, R₄ or R₅ is conjugated to D.

23. The isoflavone conjugate of claim 22, wherein a plurality of bioactive moieties (D) are conjugated to at least two of R₁, R₂, R₃, R₄ or R₅, wherein at each occurrence D may be the same or different.

24. The isoflavone conjugate of claim 22, selected from the group consisting of:
R₁ is OH, R₂ is R′CO, R₃ is OH, R₄ is OCH₃ and R₅ is H [6-carboxymethyl biochanin A];
R₁ is OH, R₂ is H, R₃ is OH, R₄ is OCH₃ and R₅ is R′CO [8-carboxymethyl biochanin A];
R₁ is O—R′CO, R₂ is H, R₃ is OH, R₄ is OH and R₅ is H [7-(O)-carboxymethyl daidzein];
R₁ is O—R′CO, R₂ is H, R₃ is H, R₄ is OCH₃ and R₅ is H [7-(O)-carboxymethyl formononetin];
R₁ is OH, R₂ is R′CO, R₃ is OH, R₄ is OH and R₅ is H [6-carboxymethyl genistein].

25. The isoflavone conjugate of claim 22, wherein D is selected from the group consisting of cytotoxic compound, cytostatic compound, antisense compound, anti-viral agent, specific antibody, biodegradable carrier, imaging and detection agents.

26. The isoflavone conjugate of claim 25, wherein D is a cytotoxic compound selected from the group consisting of: agents inhibitory of DNA synthesis and function: adriamycin, bleomycin chlorambucil cisplatin daunomycin and melphalan; agent inhibitory of microtubule (mitotic spindle) formation and function: vinblastine, vincristine, vinorelbine, paclitaxel (taxol) and docetaxel; anti metabolites: cytarabine, fluorouracil, fluroximidine, mercaptopurine, methotorexate, gemcitabin and thioquanine; alkylating agents: mechlorethamine, chlorambucil, cyclophosphamide, melphalan ifosfamide and methotrexate; antibiotics: bleomycin and mitomycin; nitrosoureas: carmustine (BCNU) and lomustine; inorganic ions: carboplatin, oxaloplatin; interferon and asparaginase; hormones: tamoxifen, leuprolide, flutamide and megestrol acetate.

27. The isoflavone conjugate of claim 25, wherein D is an agent inhibitory of DNA synthesis and function selected from the group consisting of adriamycin, bleomycin, chlorambucil, cisplatin, daunomycin and melphalan.

28. The isoflavone conjugate of claim 25, wherein D is daunomycin.

29. The isoflavone conjugate of claim 25, wherein at least one D is a biodegradable carrier.

30. The isoflavone conjugate of claim 29, wherein D is a polyvalent natural or synthetic peptide or polypeptide.

31. The isoflavone conjugate of claim 25, wherein D is an imaging agent selected from the group consisting of paramagnetic particles: gadolinium, yttrium, lutetium and gallinum; radioactive moieties: radioactive indium, rhenium and technetium; and fluorescent dyes: fluorescein isothiocyanate (FITC), green fluorescent protein (GFP), Cyan fluorescent protein (CFP), rhodamine I, II, III and IV, rhodamine B and rosamine.

32. A pharmaceutical composition comprising as an active ingredient an isoflavone conjugate having the general formula (II) 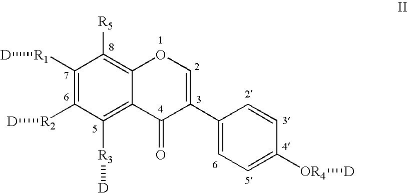
wherein
R₁ is selected from the group consisting of OH, OCH₃ OGlc, OR′COOX and OR′CO;
R₂ is selected from the group consisting of H, R′COOX and R′CO;
R₃ is selected from the group consisting of H, OH, R′COOX, R′CO, OR′COOX and OR′CO;
R₄ is selected from the group consisting of H, CH₃, R′COOX and R′CO;
R₅ is selected from the group consisting of H, R′COOX and R′CO;
D may be absent or is a bioactive moiety;
R′ is selected from the group consisting of (C₁-C₆)alkyl, (C₁-C₂₀)alkoxy, (C₁-C₂₀)alkenyl;
X is selected from the group consisting of H and (CH₂)n-Y wherein Y is CH₃ or NH₂ and n=0-10;
with the proviso that at least one of R₁, R₂, R₃, R₄ or R₅ is conjugated to D, further comprising a pharmaceutically acceptable diluent or carrier.

33. The pharmaceutical composition of claim 32 wherein a plurality of bioactive moieties (D) are conjugated to at least two of R₁, R₂, R₃, R₄ or R₅, wherein at each occurrence D may be the same or different.

34. The pharmaceutical composition of claim 32, wherein the isoflavone conjugate is selected from the group consisting of:
R₁ is OH, R₂ is R′CO, R₃ is OH, R₄ is OCH₃ and R₅ is H [6-carboxymethyl biochanin A];
R₁ is OH, R₂ is H, R₃ is OH, R₄ is OCH₃ and R₅ is R′CO [8-carboxymethyl biochanin A);
R₁ is O—R′CO, R₂ is H, R₃ is OH, R₄ is OH and R₅ is H [7-(O)-carboxymethyl daidzein];
R₁ is O—R′CO, R₂ is H, R₃ is H, R₄ is OCH₃ and R₅ is H (7-(O)-carboxymethyl formononetin];
R₁ is OH, R₂ is R′CO, R₃ is OH, R₄ is OH and R₅ is H [6-carboxymethyl genistein].

35. The pharmaceutical composition of claim 32, wherein D is selected from the group consisting of: cytotoxic compound, cytostatic compound, antisense compound, anti-viral agent, specific antibody, an imaging and detection agents and a biodegradable carrier.

36. The pharmaceutical composition of claim 35 wherein D is a cytotoxic compound selected from the group consisting of: agents inhibitory of DNA synthesis and function: adriamycin, bleomycin chlorambucil cisplatin daunomycin and melphalan; agent inhibitory of microtubule (mitotic spindle) formation and function: vinblastine, vincristine, vinorelbine, paclitaxel (taxol) and docetaxel; anti metabolites: cytarabine, fluorouracil, fluroximidine, mercaptopurine, methotorexate, gemcitabin and thioquanine; alkylating agents: mechlorethamine, chlorambucil, cyclophosphamide, melphalan ifosfamide and methotrexate; antibiotics: bleomycin and mitomycin; nitrosoureas: carmustine (BCNU) and lomustine; inorganic ions: carboplatin, oxaloplatin; interferon and asparaginase; hormones: tamoxifen, leuprolide, flutamide and megestrol acetate.

37. The pharmaceutical composition of claim 35 wherein D is an agent inhibitory of DNA synthesis and function selected from the group consisting of: adriamycin, bleomycin chlorambucil cisplatin daunomycin and melphalan.

38. The pharmaceutical composition of claim 35 wherein D is daunomycin.

39. The pharmaceutical composition of claim 35 wherein at least one D is a biodegradable carrier.

40. The pharmaceutical composition of claim 39 wherein D is a polyvalent natural or synthetic peptide or polypeptide.

41. The pharmaceutical composition of claim 35 wherein D is an imaging and detection agent selected from the group consisting of: paramagnetic particles: gadolinium, yttrium, lutetium and gallinum; radioactive moieties: radioactive indium, rhenium and technetium; and fluorescent dyes: fluorescein isothiocyanate (FITC), green fluorescent protein (GFP), Cyan fluorescent protein (CFP), rhodamine I, II, III and IV, rhodamine B and rosamine.

42. The pharmaceutical composition of claim 32 formulated for parenteral or oral administration.

43. The pharmaceutical composition of claim 42 wherein the formulation for parenteral administration is suitable for intravenous injections, intravenous infusion, intradermal, intralesional, intramuscular and subcutaneous injections or depots, or for administering laparascopically and intravesicularly.

44. The pharmaceutical composition of claim 42 wherein the formulation for oral administration is selected from liquids, suspensions, slurries, syrups, gels, tablets, pills, dragees and capsules.

45. A method for treating a subject in need thereof comprising administering a pharmaceutical composition comprising a therapeutically effective amount of an isoflavone conjugate having the general formula (II): 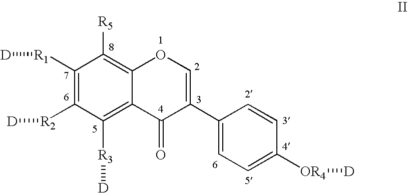
wherein
R₁ is selected from the group consisting of OH, OCH₃ OGlc, OR′COOX and OR′CO;
R₂ is selected from the group consisting of H, R′COOX and R′CO;
R₃ is selected from the group consisting of H, OH, R′COOX, R′CO, OR′COOX and OR′CO;
R₄ is selected from the group consisting of H, CH₃, R′COOX and R′CO;
R₅ is selected from the group consisting of H, R′COOX and R′CO;
D may be absent or is a bioactive moiety;
R′ is selected from the group consisting of (C₁-C₆)alkyl, (C₁-C₂₀)alkoxy, (C₁-C₂₀)alkenyl;
X is selected from the group consisting of H and (CH₂)n-Y wherein Y is CH₃ or NH₂ and n=0-10;
with the proviso that at least one of R₁, R₂, R₃, R₄ or R₅ is conjugated to D, further comprising a pharmaceutically acceptable diluent or carrier.

46. The method of claim 45 wherein a plurality of bioactive moieties (D) are conjugated to at least two of R₁, R₂, R₃, R₄ or R₅, wherein at each occurrence D may be the same or different.

47. The method of claim 45, wherein the isoflavone conjugate is selected from the group consisting of the following configurations:
R₁ is OH, R₂ is R′CO, R₃ is OH, R₄ is OCH₃ and R₅ is H [6-carboxymethyl biochanin A];
R₁ is OH, R₂ is H, R₃ is OH, R₄ is OCH₃ and R₅ is R′CO [8-carboxymethyl biochanin A];
R₁ is O—R′CO, R₂ is H, R₃ is OH, R₄ is OH and R₅ is H [7-(O)-carboxymethyl daidzein];
R₁ is O—R′CO, R₂ is H, R₃ is H, R₄ is OCH₃ and R₅ is H [7-(O)-carboxymethyl formononetin];
R₁ is OH, R₂ is R′CO, R₃ is OH, R₄ is OH and R₅ is H [6-carboxymethyl genistein].

48. The method of claim 45, wherein D is selected from the group consisting of: cytotoxic compound, cytostatic compound, antisense compound, anti-viral agent, specific antibody and biodegradable carrier.

49. The method of claim 48 wherein D is a cytotoxic compound selected from the group consisting of: agents inhibitory of DNA synthesis and function: adriamycin, bleomycin chlorambucil cisplatin daunomycin and melphalan; agent inhibitory of microtubule (mitotic spindle) formation and function: vinblastine, vincristine, vinorelbine, paclitaxel (taxol) and docetaxel; anti metabolites: cytarabine, fluorouracil, fluroximidine, mercaptopurine, methotorexate, gemcitabin and thioquanine; alkylating agents: mechlorethamine, chlorambucil, cyclophosphamide, melphalan ifosfamide and methotrexate; antibiotics: bleomycin and mitomycin; nitrosoureas: carmustine (BCNU) and lomustine; inorganic ions: carboplatin, oxaloplatin; interferon and asparaginase; hormones: tamoxifen, leuprolide, flutamide and megestrol acetate.

50. The method of claim 48 wherein D is an agent inhibitory of DNA synthesis and function selected from the group consisting of adriamycin, bleomycin chlorambucil, cisplatin, daunomycin, and melphalan.

51. The method of claim 48 wherein D is daunomycin.

52. The method of claim 48 wherein at least one D is a biodegradable carrier.

53. The method of claim 49 wherein D is a polyvalent natural or synthetic peptide or polypeptide.

54. A method for diagnosing a subject in need thereof comprising administering a pharmaceutical composition comprising a diagnostically effective amount of an isoflavone conjugate having the general formula (II): 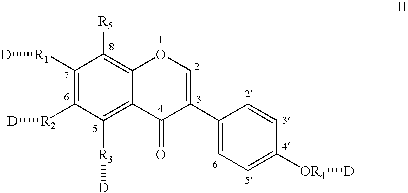
wherein
R₁ is selected from the group consisting of OH, OCH₃ OGlc, OR′COOX and OR′CO;
R₂ is selected from the group consisting of H, R′COOX and R′CO;
R₃ is selected from the group consisting of H, OH, R′COOX, R′CO, OR′COOX and OR′CO;
R₄ is selected from the group consisting of H, CH₃, R′COOX and R′CO;
R₅ is selected from the group consisting of H, R′COOX and R′CO;
D may be absent or is a bioactive moiety;
R′ is selected from the group consisting of (C₁-C₆)alkyl, (C₁-C₂₀)alkoxy, (C₁-C₂₀)alkenyl;
X is selected from the group consisting of H and (CH₂)n-Y wherein Y is CH₃ or NH₂ and n=0-10;
with the proviso that at least one of R₁, R₂, R₃, R₄ or R₅ is conjugated to D, further comprising a pharmaceutically acceptable diluent or carrier.

55. The method of claim 54 wherein the isoflavone conjugate is selected from the group consisting of the following configurations:
R₁ is OH, R₂ is R′CO, R₃ is OH, R₄ is OCH₃ and R₅ is H [6-carboxymethyl biochanin A];
R₁ is OH, R₂ is H, R₃ is OH, R₄ is OCH₃ and R₅ is R′CO [8-carboxymethyl biochanin A];
R₁ is O—R′CO, R₂ is H, R₃ is OH, R₄ is OH and R₅ is H [7-(O)-carboxymethyl daidzein];
R₁ is O—R′CO, R₂ is H, R₃ is H, R₄ is OCH₃ and R₅ is H [7-(O)-carboxymethyl formononetin];
R₁ is OH, R₂ is R′CO, R₃ is OH, R₄ is OH and R₅ is H [6-carboxymethyl genistein].

56. The method of claim 54, wherein D is an imaging and detection agent selected from the group consisting of: paramagnetic particles: gadolinium, yttrium, lutetium and gallinum; radioactive moieties: radioactive indium, rhenium and technetium; and fluorescent dyes: fluorescein isothiocyanate (FITC), green fluorescent protein (GFP), Cyan fluorescent protein (CFP), rhodamine I, II, III and IV, rhodamine B and rosamine.

57. The method of claim 56, for diagnosing or treating a disorder selected from the group consisting of cancer, cardiovascular diseases, osteoporosis, Alzheimer's disease and arteriosclerosis.

58. The method of claims 57, wherein diagnosing or treating is targeted to an estrogen receptor.

59. The method of claim 58, wherein diagnosing or treating is targeted to an estrogen receptor subtype β.
